(19) United States
(12) Patent Application Publication
(10) Pub. No.: US 2023/0192786 A1
(43) Pub. Date: Jun. 22, 2023

(54) STAT3 INHIBITION FOR TREATMENT AND PREVENTION OF HUMAN CORONAVIRUS INFECTION

(71) Applicant: GLG PHARMA, LLC, TAMPA, FL (US)

(72) Inventors: RICHARD GABRIEL, SWAMPSCOTT, MA (US); HECTOR J. GOMEZ, SALEM, MA (US)

(21) Appl. No.: 17/998,862

(22) PCT Filed: May 14, 2021
(86) PCT No.: PCT/US21/70561

§ 371 Date: Nov. 15, 2022

Related U.S. Application Data
(60) Provisional application No. 63/025,719, filed on May 15, 2020.

(51) Int. Cl. C07K 14/47; A61K 45/06

(52) U.S. Cl. CPC C07K 14/4703; A61K 45/06

(57) ABSTRACT

The subject invention pertains to the use of STAT3 inhibitors for the treatment or prevention of human coronavirus infections, such as SARS-CoV-2 or SARS-CoV-2 variant infections. Aspects of the invention include methods for treating or preventing coronavirus infection, or a symptom thereof, by administering one or more STAT3 inhibitors, such as GLG-305 or GLG-805, or a pharmaceutically acceptable salt, derivative, or prodrug thereof, to a human subject and, optionally, one or more RNA kinase inhibitors, one or more IL-6 inhibitors and/or one or more Janus kinases (JAK) inhibitors. The subject invention further pertains to pharmaceutical compositions, packaged dosage formulations, and kits for treating or preventing human coronavirus infection.

### CROSS-REFERENCE TO RELATED APPLICATION

The present application claims the benefit of U.S. Provisional Application Ser. No. 63/025,719, filed May 15, 2020, which is hereby incorporated by reference herein in its entirety, including any figures, tables, nucleic acid sequences, amino acid sequences, or drawings.

### BACKGROUND OF THE INVENTION

Coronaviruses are enveloped viruses of the family Coronaviridae, which are transmitted through the air and primarily infect the cells of the upper respiratory and gastrointestinal tract of mammals and birds. The name coronavirus is derived from the Latin “crown” or “halo”, which refers to its characteristic morphology, which resembles a crown or solar corona when imaged using an electron microscope.

Coronaviruses cause illness in adults and children ranging from the common cold to more severe diseases. Common signs of infection include respiratory symptoms, fever, cough, shortness of breath, and breathing difficulties. In more severe cases, coronavirus infection can cause pneumonia, severe acute respiratory syndrome (SARS), kidney failure, and even death. The widely publicized human coronavirus discovered in 2003, SARS-CoV, causes both upper and lower respiratory tract infections.

Coronaviruses are zoonotic, meaning they are transmitted between animals and humans. Several known coronaviruses are circulating in animals that have not yet infected humans. A novel coronavirus (nCoV) is a new strain that has not been previously identified in humans. There are currently no vaccines or antiviral drugs to prevent or treat human coronavirus infections.

Following the outbreak of SARS in 2003, which had begun the prior year in Asia, and secondary cases elsewhere in the world, the World Health Organization (WHO) issued a press release stating that a novel coronavirus, which as identified by a number of laboratories, was the causative agent for SARS. The virus was officially named the SARS coronavirus (SARS-CoV). In September 2012, a new type of coronavirus was identified, initially called Novel Coronavirus 2012, and now officially named Middle East respiratory syndrome coronavirus (MERS-CoV). In May 2014, two United States cases of MERS-CoV infection were recorded, both occurring in healthcare workers who worked in Saudi Arabia and then traveled to the U.S. In May 2015, an outbreak of MERS-CoV occurred in the Republic of Korea, when a man who had traveled to the Middle East, visited hospitals in the Seoul area to treat his illness, causing one of the largest outbreaks of MERS-CoV outside the Middle East.

In December 2019, a pneumonia outbreak was reported in Wuhan, China, and was traced to a novel strain of coronavirus, which was given the interim name 2019-nCoV by the World Health Organization (WHO), later renamed SARS-CoV-2 by the International Committee on Taxonomy of Viruses (Zhu N. et al, “A Novel Coronavirus from Patients with Pneumonia in China, 2019”, N Engl J Med, February 2020, 382(8):727-733, Epub 24 Jan. 2020). Coronavirus disease 2019 (COVID-19) is the infectious disease caused by SARS-CoV-2. SARS-CoV-2 has killed more people than 2003 SARS outbreak and is rapidly spreading in multiple waves of new variants in India, Pakistan, and Brazil in 2021, infecting millions of people per day and causing healthcare systems to collapse.

A new virus variant has one or more genetic distinctions (e.g., mutations) that differentiate it from the wild-type or predominant virus variants already circulating among the general population, and several variants of SARS-CoV-2 have been reported in the United States and globally. Some of the SARS-CoV-2 variants that have emerged are particularly concerning, including at least B.1.1.7 identified in the United Kingdom, B.1.351 identified in South Africa, and P.1 identified in travelers from Brazil.

There remains a need for a safe and effective method of treating or preventing infections of coronavirus and associated symptoms, in particular those related to COVID-19, and its causative agent, SARS-CoV-2, and its variants.

### BRIEF SUMMARY OF THE INVENTION

The present invention concerns the use of Signal Transduction and Activator of Transcription 3 (STAT3) inhibitors for the treatment or prevention of human coronavirus infections, such as SARS-CoV-2 and its variants. In some embodiments, the STAT3 inhibitor is administered with one or more additional agents, such as JAK inhibitors (e.g., Baricitinib), RNA virus inhibitors (e.g., remdesivir or favipiravir), and/or IL-6 inhibitors.

One aspect of the invention is a method for the treatment or prevention of human coronavirus infection, comprising administering a STAT3 inhibitor to a human subject in need thereof. In some embodiments, the STAT3 inhibitor is administered to a human subject infected by a human coronavirus, such as SARS-CoV-2, as therapy. In some embodiments, the subject has the disease COVID-19. In other embodiments, the STAT3 inhibitor is administered to a human subject not infected by human coronavirus, such as SARS-CoV-2, as prophylaxis (to prevent or delay the onset of human coronavirus infection).

In particular embodiments, the human subject is symptomatic for the coronavirus infection (e.g., SARS-CoV-2 infection) at the time of the administration. In other embodiments, the human subject has tested positive for the coronavirus infection at the time of the administration but is asymptomatic. In further embodiments, the method further comprises administering one or more additional agents, such as a drug, to the human subject.

In particular embodiments, the human subject is asymptomatic for the coronavirus infection and/or has been diagnosed as corona virus negative (e.g., SARS-CoV-2 negative) at the time of the administration. In other particular embodiments, the human subject has been exposed to coronavirus (e.g., SARS-CoV-2 or a variant) or has had close contact with someone infected with the coronavirus (e.g., SARS-CoV-2 or a variant). In further embodiments, the method further comprises administering one or more additional agents, such as a drug, to the human subject.

The method may include detecting the presence of the human coronavirus infection prior to administration of the STAT3 inhibitor. Any suitable method for diagnosis of COVID-19, or testing for SARS-CoV-2 or variant infection, can be used, and such methods are well known in the art, including nucleic acid assays.

Another aspect of the invention concerns a method for inhibiting a human coronavirus infection, such as SARS-CoV-2 or its variants, in a human cell, comprising contacting the cell in vitro or in vivo with a STAT3 inhibitor, or a pharmaceutically acceptable salt, derivative, or prodrug thereof, before or after the cell is infected.

Another aspect of the invention concerns a method for treating symptoms or consequences of a human coronavirus infection, comprising administering a STAT3 inhibitor to a human subject in need thereof. The symptoms may be what has been described as a cytokine storm, which can lead to further consequences such as, for example fibrosis of tissues, including lung tissues. The pathophysiology of SARS-CoV-2-induced acute respiratory distress syndrome (ARDS) has similarities to that of severe community-acquired pneumonia caused by other microorganisms. The overproduction of early response proinflammatory cytokines (tumor necrosis factor (TNF), IL-6, and IL-1β) results in the cytokine storm, leading to an increased risk of vascular hyperpermeability, multi-organ failure, and death.

In some embodiments of the methods of the invention, the STAT3 inhibitor comprises one or more of Pt-401 (GLG-401), Withacnistin (GLG-101), NSC-74859 (GLG-302), NSC-59263 (GLG-303), NSC-42067 (GLG-304), GLG-305, S3I-M2001 (GLG-202), HL2-006-1 (GLG-306), HL2-006-2 (GLG-307), Pyrimethamine (GLG-801), Pimozide (GLG-802),

Guanabenz Acetate (GLG-803), Alprenolol hydrochloride (GLG-804), Solanine alpha (GLG-806), Fluoxetine hydrochloride (GLG-807), Ifosfamide (GLG-808), Pyrvinium pamoate (GLG-809), Moricizine hydrochloride (GLG-810), 3,3′-oxybis[tetrahydrothiophene, 1,1,1′,1′-tetraoxide] (GLG-811), Nifuroxazide (GLG-812), Pyrimethamine IV formulae—methane sulfonic acid salt (GLG-805), 188-9 (TTi-101) N-{1′,2-dihydroxy-[1,2′-binaphthalen]-4′-yl}-4-methoxybenzene-1-sulfonamide, STX-0119, Napabucasin (BBI-608), Niclosamide, CPD-188, GPB730, GPA 512, WP1066, Curcumin, SBT-100, SBT-102, SBT-300, or a derivative of any of the foregoing that retains STAT3 inhibitory activity. In some embodiments of the methods of the invention, the STAT3 inhibitor comprises two or more STAT3 inhibitors.

In some embodiments of the methods of the invention, the human coronavirus is selected from among SARS-CoV-2, SARS-CoV, and MERS-CoV.

In some embodiments of the methods of the invention, the human coronavirus is a variant of SARS-CoV-2 that is a variant of interest or variant of concern. In some embodiments of the methods of the invention, the human coronavirus is a variant of concern such as the B.1.1.7 variant, B.1.351 variant, P.1 variant, B.1.427 variant, or B.1.429. In some embodiments of the methods of the invention, the human coronavirus is a variant of interest such as the 1.526 variant, B.1.526.1 variant, B.1.525 variant, or P.2 variant.

In some embodiments of the methods of the invention, the human coronavirus is a common human coronavirus, such as type 229E, NL63, OC43, and HKU1.

Another aspect of the invention concerns a composition comprising a STAT3 inhibitor for treatment of human coronavirus infection, such as such as SARS-CoV-2 infection. In some embodiments of the composition of the invention, the STAT3 inhibitor is one or more of Pt-401 (GLG-401), Withacnistin (GLG-101), NSC-74859 (GLG-302), NSC-59263 (GLG-303), NSC-42067 (GLG-304), GLG-305, S3I-M2001 (GLG-202), HL2-006-1 (GLG-306), HL2-006-2 (GLG-307), Pyrimethamine (GLG-801), Pimozide (GLG-802), Guanabenz Acetate (GLG-803), Alprenolol hydrochloride (GLG-804), Solanine alpha (GLG-806), Fluoxetine hydrochloride (GLG-807), Ifosfamide (GLG-808), Pyrvinium pamoate (GLG-809), Moricizine hydrochloride (GLG-810), 3,3′-oxybis[tetrahydrothiophene, 1,1,1′,1′-tetraoxide] (GLG-811), Nifuroxazide (GLG-812), Pyrimethamine IV formulae—methane sulfonic acid salt (GLG-805), 188-9 (TTi-101) N-{1′,2-dihydroxy-[1,2′-binaphthalen]-4′-yl}-4-methoxybenzene-1-sulfonamide, STX-0119, Napabucasin (BBI-608), Niclosamide, CPD-188, GPB730, GPA 512, WP1066, Curcumin, SBT-100, SBT-102, SBT-300, or a derivative or prodrug of any of the foregoing that has STAT3 inhibitory activity. In some embodiments of the composition of the invention, the composition comprises two or more STAT3 inhibitors. In some embodiments of the composition of the invention, the composition further comprises one or more additional agents such as a JAK inhibitor (e.g., Baricitinib), RNA virus inhibitor (e.g., remdesivir or favipiravir), and/or IL-6 inhibitor. In some embodiments of the composition, the composition comprises a packaged dosage formulation or a kit for treatment or prevention of a human coronavirus infection.

Another aspect of the invention concerns kits comprising, in one or more containers, one or more STAT3 inhibitors. A kit of the invention can also comprise one or more other agents such as compounds, biological molecules, or drugs. In one embodiment, the kit of the invention comprises one or more STAT3 inhibitors, and optionally comprises one or more additional agents, such as agents that may be useful in treating a viral infection (e.g., a human coronavirus infection, such as SARS-CoV-2 or variant).

### BRIEF DESCRIPTION OF THE DRAWINGS

FIG. 1. Chemical structure of Pt-401 (GLG-401). IUPAC name: 1,4-bis(2-hydroxypropyl)piperazine-1,4-diium; hexachloroplatinum+2 Groups.

FIG. 2. Chemical structure of Withacnistin (GLG-101). IUPAC Name: [(1S,2R,6S,7R,9R,11S,12S,15R,16R)-15-[(1S)-1-[(2R)-4,5-dimethyl-6-oxo-2,3-dihydropyran-2-yl]ethyl]-6-hydroxy-2-methyl-3-oxo-8-oxapentacyclo[9.7.0.0^{2,7}.0^{7,9}.0^{12,16}]octadec-4-en-16-yl]methyl acetate.

FIG. 3. Chemical structure of NSC-74859 (GLG-302). IUPAC name: 2-hydroxy-4-{2-[(4-methylbenzenesulfonyl)oxy]acetamido}benzoic acid.

FIG. 4. Chemical structure of NSC-59263 (GLG-303). IUPAC name: 3-benzoyloxy)-4,5-dihydroxybenzoic acid.

FIG. 5. Chemical structure of NSC-42067 (GLG-304). IUPAC name: 3-(2-{[(6,8-disulfonaphthalen-2-yl)amino]methyl}-3,4,5-trihydroxybenzoyloxy)-4,5-dihydroxybenzoic acid.

FIG. 6. Chemical structure of GLG-305 (Shoemaker et al., International Publication No. WO 2018/187551, Oct. 11, 2018). IUPAC name: 2-hydroxy-4-[[2-[[(4-methylphenyl)sulfonyl]oxy]acetyl]amino]benzoic acid.

FIG. 7. Chemical structure of 531-M2001 (GLG-202) (Turkson et al., U.S. Pat. No. 8,609,639). IUPAC name: [(4-{[4-(hexylcarbamoyl)-2-(naphthalen-1-yl)-1,3-oxazol-5-yl]methyl}phenyl)methyl]phosphonic acid.

FIG. 8. Chemical structure of HL2-006-1 (GLG-306) (Turkson et al., U.S. Patent Application Publication No. 2017/0202795). IUPAC name: 4-(3-{[1,1′-biphenyl]-4-sulfonamido}-2-oxopropyl)-2-hydroxybenzoic acid.

FIG. 9. Chemical structure of HL2-006-2 (GLG-307) (Turkson et al., U.S. Patent Application Publication No. 2017/0202795). IUPAC name: 4-[2-(benzyloxy)acetamido]benzoic acid.

FIG. 10. Chemical structure of Pyrimethamine (GLG-801) (Frank, U.S. Patent Application Publication No. 2011/0144043). IUPAC name: 5-(4-Chlorophenyl)-6-ethyl-2,4-pyrimidinediamine; 5-(4-chlorophenyl)-6-ethylpyrimidine-2,4-diamine.

FIG. 11. Chemical structure of Pimozide (GLG-802) (Frank, U.S. Patent Application Publication No. 2011/0144043). IUPAC name: 1-{1-[4,4-bis(4-fluorophenyl)butyl]piperidin-4-yl}-2,3-dihydro-1H-1,3-benzodiazol-2-one.

FIG. 12. Chemical structure of Guanabenz Acetate (GLG-803) (Frank, U.S. Patent Application Publication No. 2011/0144043). IUPAC name: N″-[(E)-[(2,6-dichlorophenyl)methylidene]amino]guanidine acetate.

FIG. 13. Chemical structure of Alprenolol hydrochloride (GLG-804) (Frank, U.S. Patent Application Publication No. 2011/0144043). IUPAC name: 1-(o-Allylphenoxy)-3-(isopropylamino)-2-propanol hydrochloride.

FIG. 14. Chemical structure of Solanine alpha (GLG-806) (Frank, U.S. Patent Application Publication No. 2011/0144043). IUPAC name: (2S,3S,4S,5R,6R)-2-{[(2R,3S,4S,5S,6R)-4-{[(2S,4R,5R,6R)-4,5-dihydroxy-6-(hydroxymethyl)oxan-2-yl]oxy}-5-hydroxy-6-(hydroxymethyl)-2-{[(2S,7S,10R,11R,15R,16R,17S,20R,23R)-10,16,20-trimethyl-22-azahexacyclo[12.10.0.0^{2,11}.0^{5,10}.0^{15,23}.0^{17,22}]tetracos-4-en-7-yl]oxy}oxan-3-yl]oxy}-6-methyloxane-3,4,5-triol.

FIG. 15. Chemical structure of Fluoxetine hydrochloride (GLG-807) (Frank, U.S. Patent Application Publication No. 2011/0144043). IUPAC name: methyl({3-phenyl-3-[4-(trifluoromethyl)phenoxy]propyl})amine.

FIG. 16. Chemical structure of Ifosfamide (GLG-808) (Frank, U.S. Patent Application No. 2011/0144043). IUPAC name: 3-(2-chloroethyl)-2-[(2-chloroethyl)amino]-1,3,2lambda5-oxazaphosphinan-2-one.

FIG. 17. Chemical structure of Pyrvinium pamoate (GLG-809) (Frank, U.S. Patent Application Publication No. 2011/0144043). IUPAC name: 2-[(1E)-2-(2,5-dimethyl-1-phenyl-1H-pyrrol-3-yl)ethenyl]-5-(dimethylamino)-1-methyl-3H-indol-1-ium; 4-[(4-carboxy-3-hydroxynaphthalen-2-yl)methyl]-3-hydroxynaphthalene-2-carboxylic acid.

FIG. 18. Chemical structure of Moricizine hydrochloride (GLG-810) (Frank, U.S. Patent Application Publication No. 2011/0144043). IUPAC name: ethyl N-{10-[3-(morpholin-4-yl)propanoyl]-10H-phenothiazin-2-yl}carbamate.

FIG. 19. Chemical structure of 3,3′-oxybis[tetrahydrothiophene, 1,1,1′,1′-tetraoxide] (GLG-811) (Frank, U.S. Patent Application Publication No. 2011/0144043). IUPAC name: 3-[(1,1-dioxo-1lambda6-thiolan-3-yl)oxy]-1lambda6-thiolane-1,1-dione.

FIG. 20. Chemical structure of Nifuroxazide (GLG-812) (Frank, U.S. Patent Application Publication No. 2011/0144043). IUPAC name: N-hydroxy-5-[(E)-{[(4-hydroxyphenyl)formamido]imino}methyl]-N-oxofuran-2-aminium.

FIG. 21. Chemical structure of Pyrimethamine IV formulae—methane sulfonic acid salt (GLG-805) (Kaczmarek et al., International Publication No. WO 2019/182463). IUPAC name: 5-(4-chlorophenyl)-6-ethyl-2,4-pyrimidinediamine methanesulfonic acid salt.

FIG. 22. Chemical structure of 188-9 (TTi-101) N-{1′,2-dihydroxy-[1,2′-binaphthalen]-4′-yl}-4-methoxybenzene-1-sulfonamide (Tweardy et al., U.S. Patent Application Publication No. 2014/0296270). IUPAC name: N-{1′,2-dihydroxy-[1,2′-binaphthalen]-4′-yl}-4-methoxybenzene-1-sulfonamide; U.S. Patent Application Publication No. 2014/0296270.

FIG. 23. Chemical structure of STX-0119. IUPAC name: N-{[2,2′-bifuran]-5-yl}-2-phenylquinoline-4-carboxamide.

FIG. 24. Chemical structure of Napabucasin (BBI-608). IUPAC name: 2-acetyl-4H,9H-naphtho[2,3-b]furan-4,9-dione.

FIG. 25. Chemical structure of Niclosamide. IUPAC name: 3-chloro-4-(5-chloro-2-hydroxybenzamido)-N-hydroxy-N-oxoanilinium.

FIG. 26. Chemical structure of CPD-188. IUPAC name: 4-({3-[(carboxymethyl)sulfanyl]-4-hydroxynaphthalen-1-yl}methanesulfonyl)benzoic acid.

FIG. 27. Chemical structure of GPB730. IUPAC name: (2S)-N-[(2E,4E,6S,7R)-7-{(2S,3S,4R,5R)-3,4-Dihydroxy-5-[(1E,3E,5E)-7-(2-hydroxy-4-oxo-1,4-dihydro-3-pyridinyl)-6-methyl-7-oxo-1,3,5-heptatrien-1-yl]tetrahydro-2-furanyl}-6-methoxy-5-methyl-2,4-octadien-1-yl]-2-{(2R,3R,4R,6 S)-2,3,4-trihydroxy-5,5-dimethyl-6-[(1E,3E)-1,3-pentadien-1-yl]tetrahydro-2H-pyran-2-yl}butanamide.

FIG. 28. Chemical structure of GPA 512. IUPAC name: methyl 2-acetamido-3-{[(1S,4R,7R,9S,11S)-11-hydroxy-9-methyl-2-oxo-3-oxatricyclo[5.3.1.0^{4,11}]undecan-10-yl]sulfanyl}propanoate.

FIG. 29. Chemical structure of WP1066. IUPAC name: (2E)-3-(6-bromopyridin-2-yl)-2-cyano-N-[(1S)-1-phenylethyl]prop-2-enamide.

FIG. 30. Chemical structure of Curcumin. IUPAC names: (1E,4Z,6E)-5-hydroxy-7-(4-hydroxy-3-methoxyphenyl)-1-(3-methoxy-4-methylphenyl)hepta-1,4,6-trien-3-one and (1E,6E)-1-(4-hydroxy-3-methoxyphenyl)-7-(3-methoxy-4-methylphenyl)hepta-1,6-diene-3,5-dione.

### DETAILED DISCLOSURE OF THE INVENTION

An aspect of the invention concerns a method for treatment or prevention of a human coronavirus infection, such as SARS-CoV-2, in a human subject, comprising administering to the human subject an effective amount of STAT3 inhibitors, such as GLG-305 or GLG-805, or a pharmaceutically acceptable salt, derivative, or prodrug thereof. The STAT3 inhibitor may be administered to the human subject before or after initiation of the coronavirus infection, thereby treating the coronavirus infection. In some embodiments, the subject has the disease COVID-19 at the time that one or more STAT3 inhibitor(s) is/are administered.

In certain embodiments, the STAT3 inhibitor can be administered concurrently with other agents, such as one or more RNA virus inhibitors, one or more IL-6 inhibitors, and/or one or more Janus kinase (JAK) inhibitors. Additionally, the JAK inhibitor, IL-6 inhibitor, or RNA virus inhibitor can be administered simultaneously, before, or after administration of the STAT3 inhibitor.

In certain embodiments, the STAT3 inhibitor can be administered after the viral infection. The STAT3 inhibitor can limit or prevent complications or symptoms of the previous infection.

In certain embodiments, the STAT3 inhibitor can be administered to treat symptoms, complications, or comorbidities of a human coronavirus infection. Complications, comorbidities, or symptoms can include a cytokine storm or fibrosis of tissues, such as, for example, lung tissue.

Another aspect of the invention concerns a method for inhibiting a human coronavirus infection in a human cell, comprising contacting the cell in vitro or in vivo with a STAT3 inhibitor, or a pharmaceutically acceptable salt, derivative, or prodrug thereof, before or after the cell is infected and, optionally, with one or more RNA virus inhibitors, one or more IL-6 inhibitors, and/or one or more JAK inhibitors.

The human coronavirus may be any time or subgroup, including alpha, beta, gamma, and delta. In some embodiments of the aforementioned methods of the invention, the human coronavirus is selected from among SARS-CoV-2 or a variant thereof, SARS-CoV, and MERS-CoV. In some embodiments of the aforementioned methods of the invention, the human coronavirus is a common human coronavirus, such as type 229E, NL63, OC43, and HKU1.

In some embodiments of the methods of the invention, the human coronavirus is a variant of SARS-CoV-2 that is a variant of interest or variant of concern. A variant of the virus is considered to be concerning when it increases the risk to human health. The risk to human health can be increased due to one or more factors. For example, the risk can be increased because a variant is able to spread more easily, cause more severe illness, escape the immune protection provided by available COVID-19 vaccines or by natural infection with the virus that causes COVID-19, make viral tests less accurate, make some treatments less effective, or any combination of two or more of the foregoing.

In some embodiments of the methods of the invention, the human coronavirus is a variant of concern such as the B.1.1.7 variant, B.1.351 variant, P.1 variant, B.1.427 variant, or B.1.429. In some embodiments of the methods of the invention, the human coronavirus is a variant of interest such as the 1.526 variant, B.1.526.1 variant, B.1.525 variant, or P.2 variant.

Another aspect of the invention concerns a composition comprising a STAT3 inhibitor, or a pharmaceutically acceptable salt, derivative, or prodrug thereof and, optionally, with one or IL-6 inhibitors, one or more RNA virus inhibitors and/or one or more JAK inhibitors.

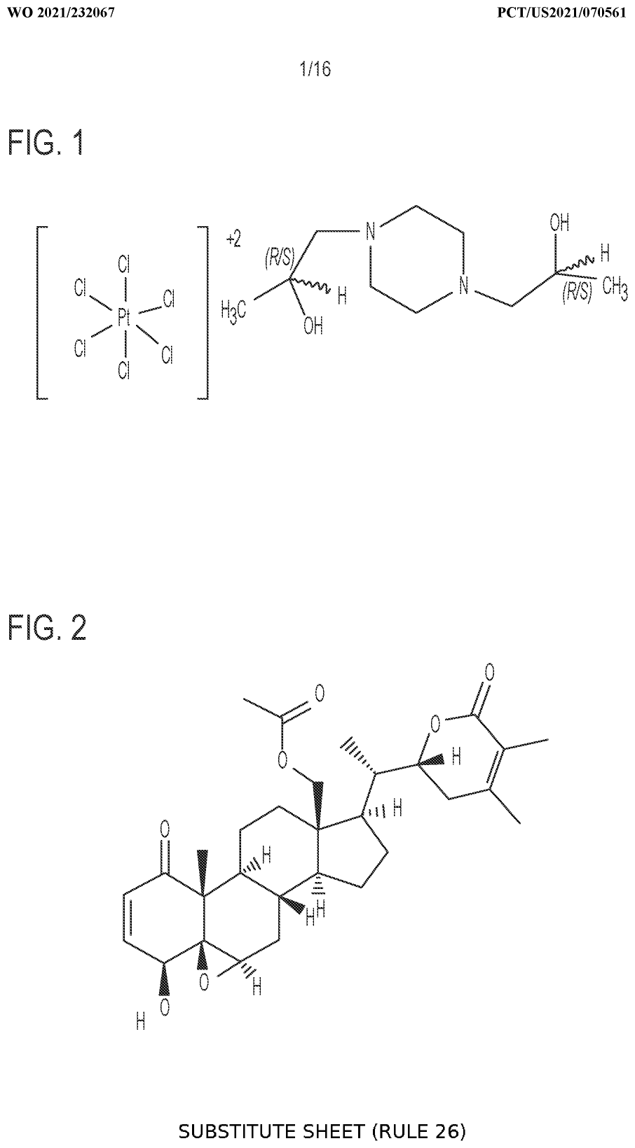

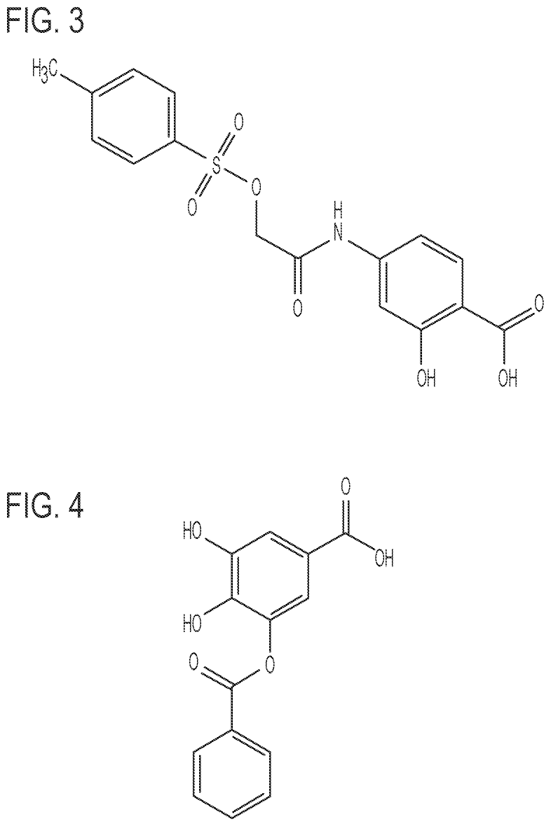

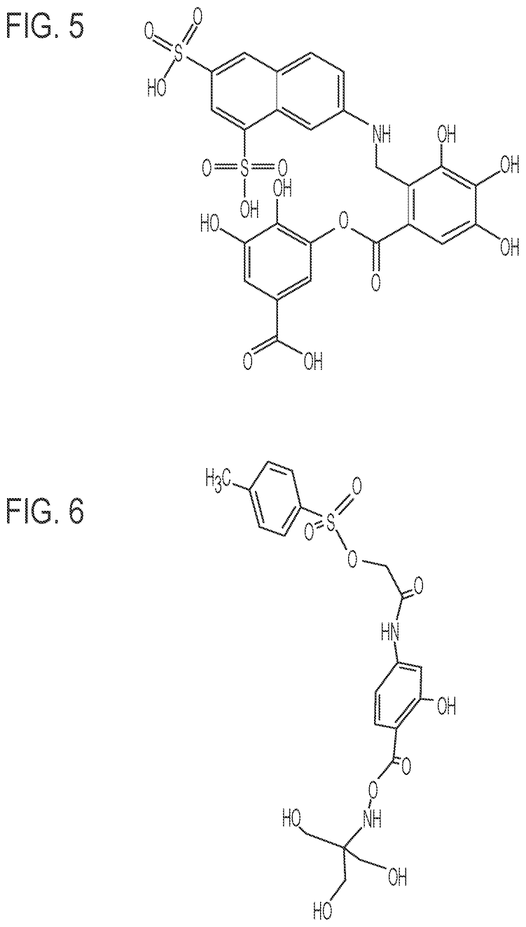

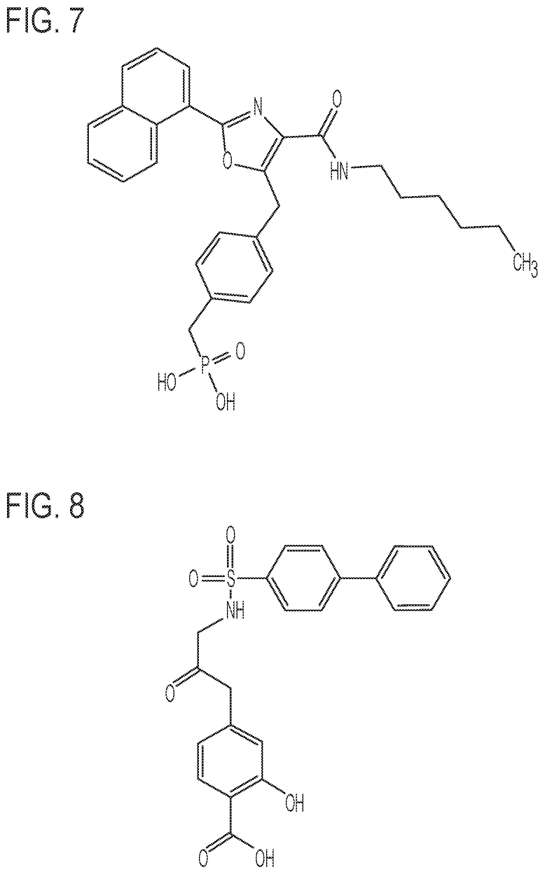

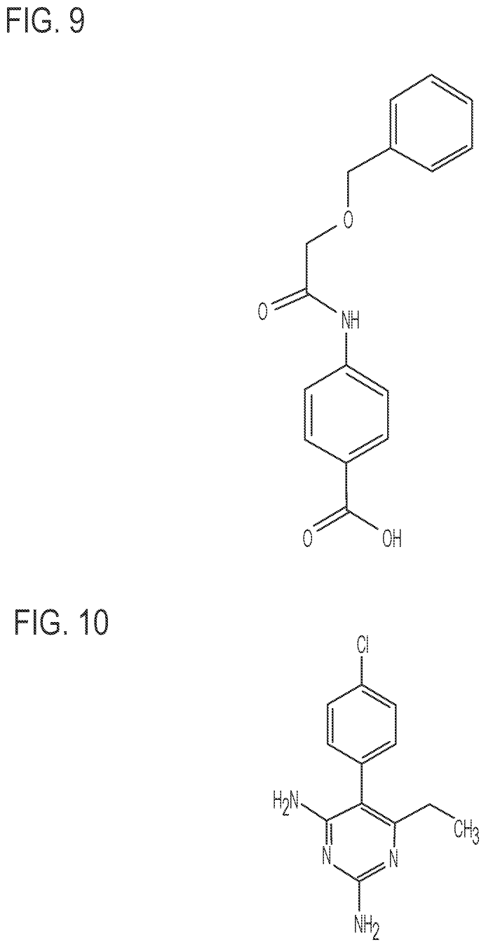

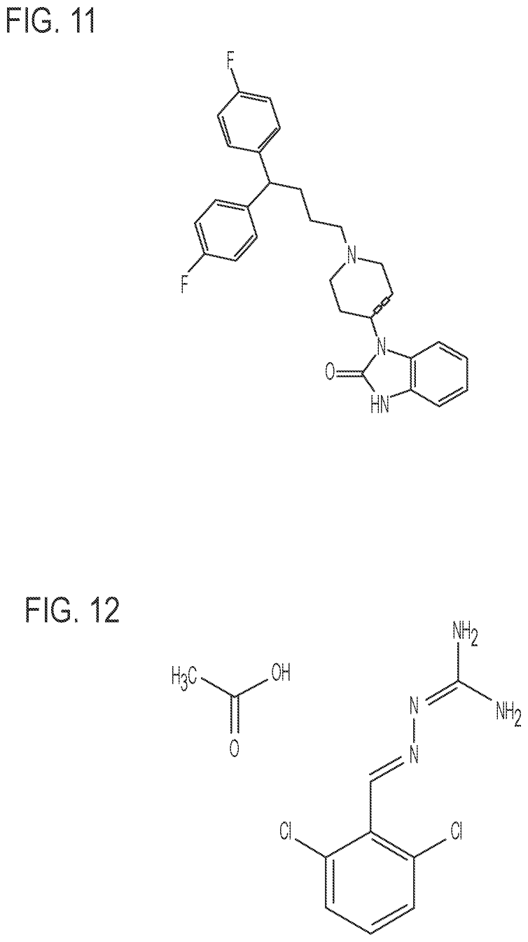

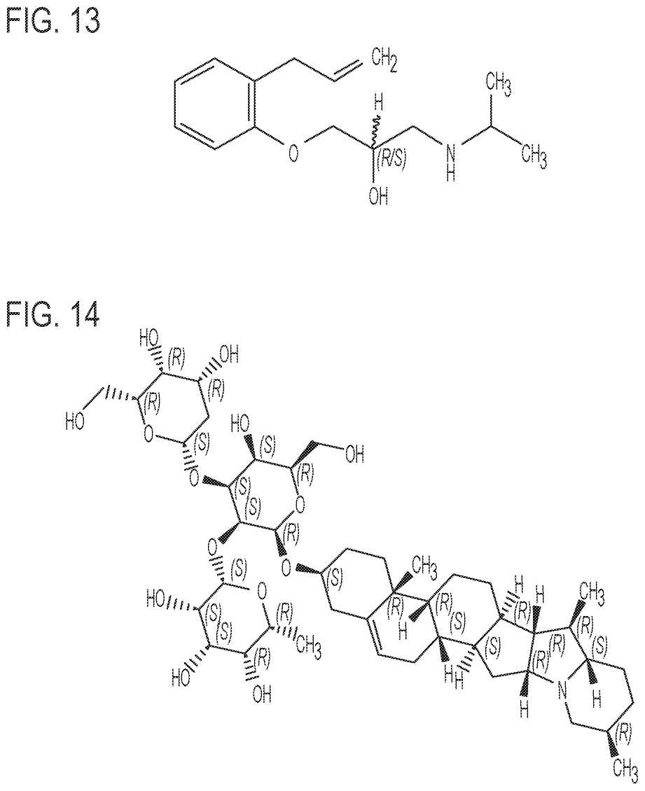

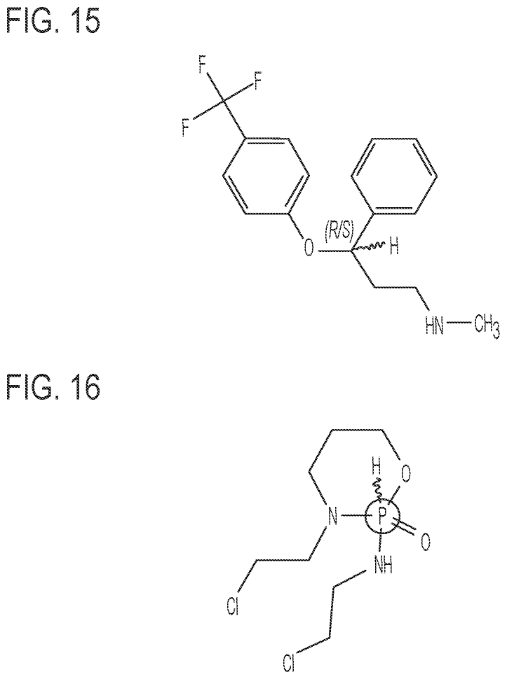

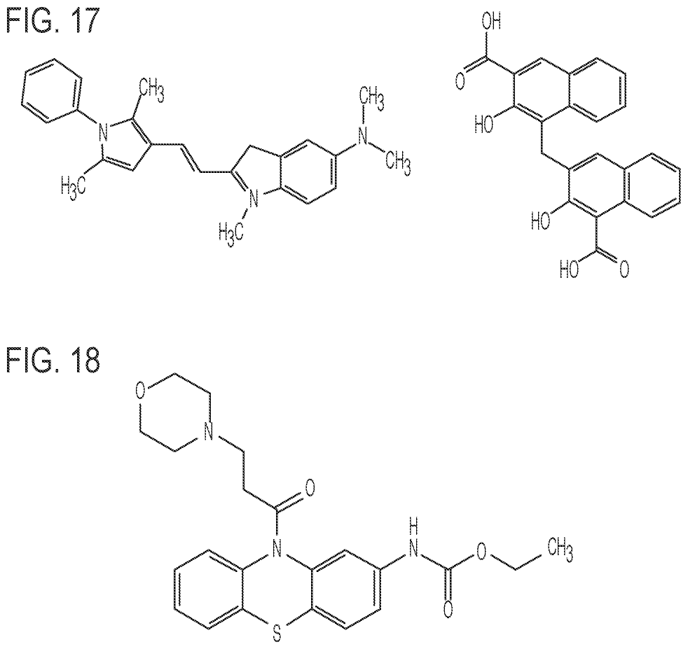

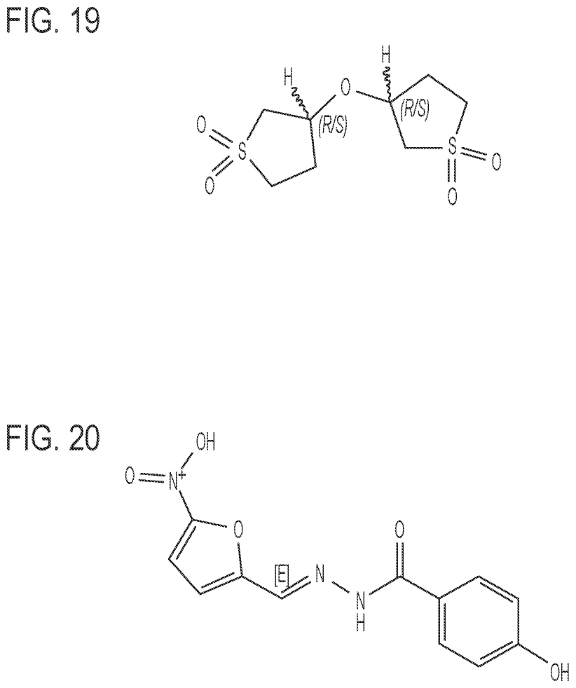

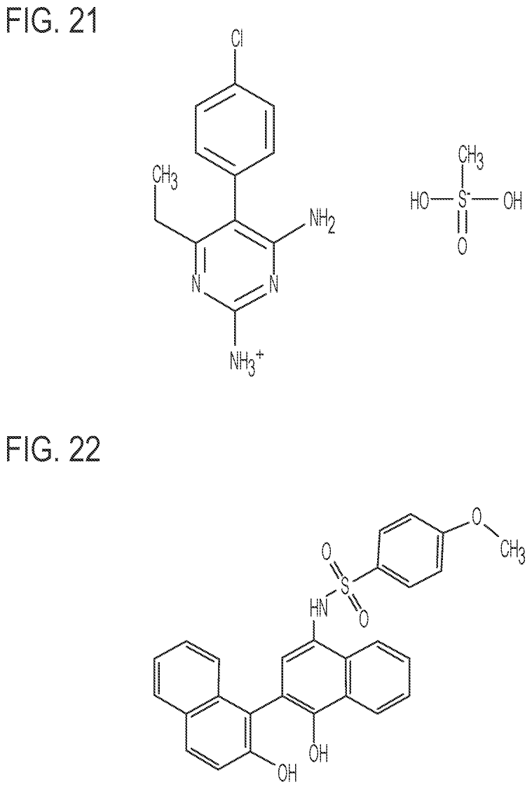

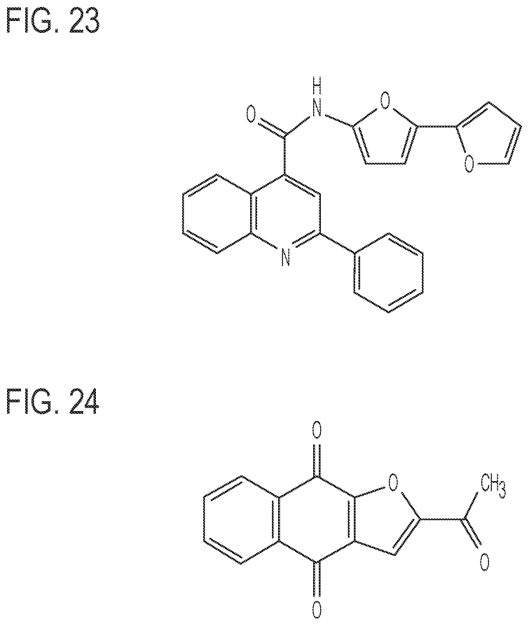

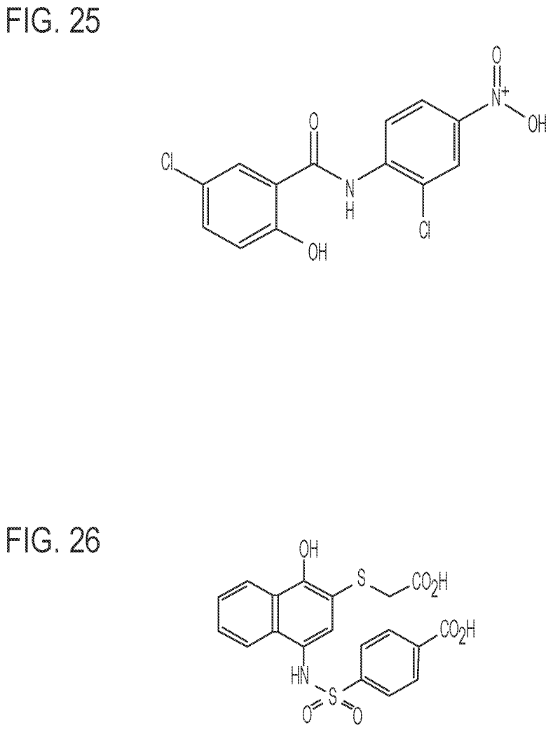

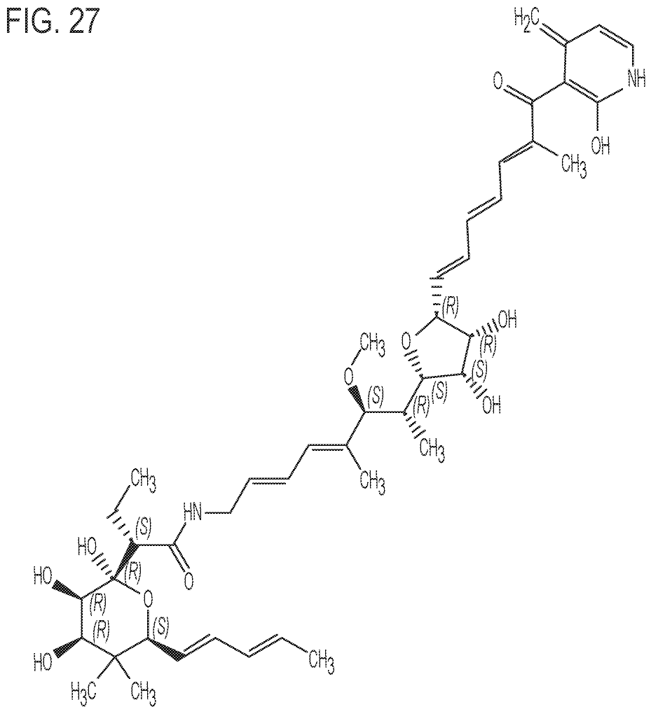

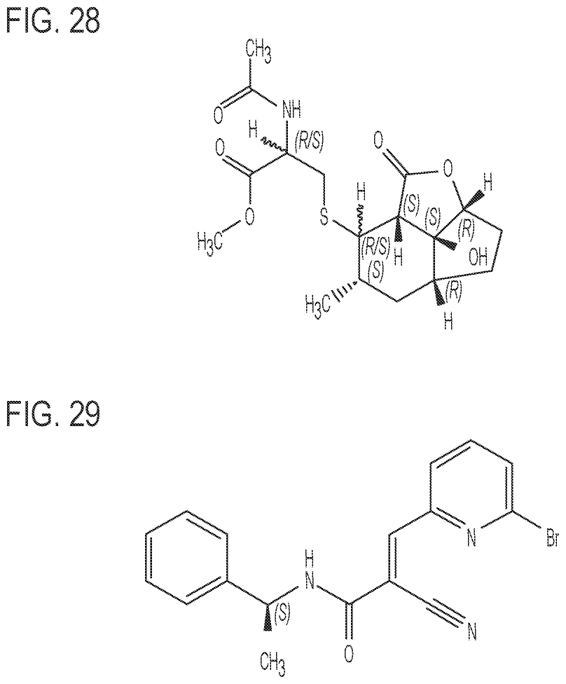

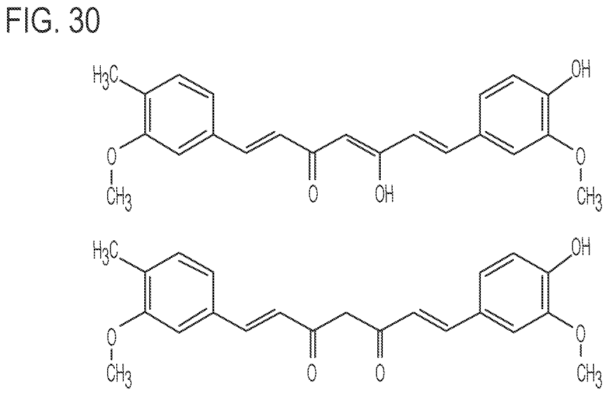

In one embodiment of the compositions and methods of the invention, the STAT3 inhibitor comprises one or more compounds disclosed herein and/or in FIGS. 1-30, or a structural or functional derivative thereof that retains STAT3 inhibitory activity, or a pharmaceutically acceptable salt of any of the foregoing.

### STAT3 Inhibitors

In certain embodiments, one or more STAT3 inhibitors are administered or are in a composition. STAT3 is involved in cell division and other crucial cell mechanisms. It is highly conserved and turned on only briefly and periodically. In diseases, when STAT3 is constitutively phosphorylated, dimerizes and crosses the nuclear membrane to initiate a cascade of subsequent events, it is often turned on nearly all the time. Extreme patient response to infection, cancer treatment, or radiation, in which the immune system is over-activated, is called acute respiratory distress syndrome (ARDS). The syndrome often leads to septic shock and multi-organ failure and death. While a variety of patient responses are evident, immune compromised individuals, elderly or individuals with compromised health conditions are at the highest risk. In ARDS, STAT3 is upregulated nearly 100%, indicating that a STAT3 inhibitor can be used in the treatment of ARDS as well as its potential treatment in combination with other medications for coronavirus, recently identified of previously approved drugs that treat SARS-CoV, MERS-CoV and may also work on COVID-19. Both SARS and MERS viruses have shown high virulence and potential responses to previously approved medications that are currently used for other indications.

In certain embodiments, the STAT3 inhibitor inhibits phosphorylation of STAT3, inhibits STAT3 activation, inhibits dimerization of STAT3, inhibits nuclear translocation of STAT3, and/or inhibits STAT3 DNA-binding.

In some embodiments, the STAT3 inhibitor inhibits phosphorylation of STAT3. In some embodiments, the STAT3 inhibitor or combination of inhibitors inhibit phosphorylation at the serine 727 residue (e.g., pyrimethamine or a pharmaceutically acceptable salt thereof, such as pyrimethamine methanesulfonate) and/or the tyrosine 705 residue (e.g., NSC-74859 (GLG-302) or GLG-305, or a pharmaceutically acceptable salt thereof).

In some embodiments, the method of the invention comprises administering a combination of STAT3 inhibitors to the subject, comprising at least one STAT3 inhibitor that inhibits phosphorylation at the serine 727 residue (e.g., pyrimethamine or a pharmaceutically acceptable salt thereof, such as pyrimethamine methanesulfonate), and at least one STAT3 inhibitor that inhibits phosphorylation at the tyrosine 705 residue (e.g., NSC-74859 (GLG-302) or GLG-305, or a pharmaceutically acceptable salt thereof). The combination of STAT3 inhibitors may be administered in the same composition, or in separate compositions simultaneously or consecutively in any order.

In some embodiments, the composition of the invention comprises a combination of two or more STAT3 inhibitors, comprising at least one STAT3 inhibitor that inhibits phosphorylation at the serine 727 residue (e.g., pyrimethamine or a pharmaceutically acceptable salt thereof, such as pyrimethamine methanesulfonate), and at least one STAT3 inhibitor that inhibits phosphorylation at the tyrosine 705 residue (e.g., NSC-74859 (GLG-302) or GLG-305, or a pharmaceutically acceptable salt thereof).

In certain embodiments, STAT3 inhibitors can have downstream affects by inhibiting the phosphorylation of STAT3. For example, inhibition of STAT3 phosphorylation can increase nitric oxide NO (nitric oxide) production and/or reduce consumption. The inhibition of the phosphorylation of serine 727 by the STAT3 inhibitor can increase NO production by activating enzymes that produce NO. Additionally, NO can inhibit viral particle production.

In certain embodiments, the STAT3 inhibitors can prevent STAT3 phosphorylation, dimerization STAT3, or DNA binding of STAT3. In preferred embodiments, the STAT3 inhibitors prevent phosphorylation, dimerization, or DNA binding at the tyrosine 705 residue and/or the serine 727 residue.

In certain embodiments, STAT3 inhibitors block the activity of STAT3. Without wishing to be bound by theory, in certain embodiments, the STAT3 inhibitors prevent a coronavirus from using STAT3 to keep a cell dividing and/or shortening the time that STAT3 is active. In certain embodiments, the STAT3 inhibitor can directly phosphorylate or dephosphorylate the tyrosine 705 residue and/or the serine 727 residue. In other embodiments, a STAT3 inhibitor can indirectly phosphorylate or dephosphorylate the tyrosine 705 residue and/or the serine 727 residue, including determining the phosphorylation and dephosphorylation patterns altered by a coronavirus. In certain embodiments, a STAT3 inhibitor activates, directly or indirectly, TC45 phosphatase.

In certain embodiments, the STAT3 inhibitor can be used to treat comorbidities, complications, symptoms, or other consequences of a human coronavirus infection. Examples of complications include a cytokine storm, in which the innate immune system release a large number of cytokines, pneumonia, viral sepsis, acute respiratory distress syndrome, kidney failure, fibrosis of tissues, and cytokine release syndrome. Examples of symptoms include fever, cough, fatigue, shortness of breath, and loss of smell and/or taste.

Examples of STAT3 inhibitors suitable for the subject invention include, but are not limited to, Pt-401 (GLG-401), Withacnistin (GLG-101), NSC-74859 (GLG-302), NSC-59263 (GLG-303), NSC-42067 (GLG-304), GLG-305, S31-M2001 (GLG-202), HL2-006-1 (GLG-306), HL2-006-2 (GLG-307), Pyrimethamine (GLG-801), Pimozide (GLG-802), Guanabenz Acetate (GLG-803), Alprenolol hydrochloride (GLG-804), Solanine alpha (GLG-806), Fluoxetine hydrochloride (GLG-807), Ifosfamide (GLG-808), Pyrvinium pamoate (GLG-809), Moricizine hydrochloride (GLG-810), 3,3′-oxybis[tetrahydrothiophene, 1,1,1′,1′-tetraoxide] (GLG-811), Nifuroxazide (GLG-812), Pyrimethamine IV formulae—methane sulfonic acid salt (GLG-805), 188-9 (TTi-101) N-{1′,2-dihydroxy-[1,2′-binaphthalen]-4′-yl}-4-methoxybenzene-1-sulfonamide, STX-0119, Napabucasin (BBI-608), Niclosamide, CPD-188, GPB730, GPA 512, WP1066, Curcumin, SBT-100, SBT-102, SBT-300, or a derivative of any of the foregoing that retains STAT3 inhibitory activity. Antibodies (monoclonal, polyclonal, or antigen-binding fragments thereof) can be used as STAT3 inhibitors, such as single domain antibodies. SBT-100, SBT-102, and SBT-300 are single domain antibodies produced by the Singh Biotechnology Company (U.S. Pub. No.: US 2016/0115247 A1 US 201601 15247A1 Singh (43) Pub. Date: Apr. 28, 2016).

In some embodiments, the STAT3 inhibitor is administered in combination with one or more additional agents, such as agents useful for treating or preventing a coronavirus infection or a symptom thereof. The one or more additional agents may be STAT3 inhibitors or non-STAT3 inhibitors, or a combination. The one or more additional agents may be, for example, one or more RNA virus inhibitors, one or more IL-6 inhibitors, and/or one or more Janus kinase (JAK) inhibitors, or another type of agent. The one or more additional agents may be administered in the same composition as the one or more STAT3 inhibitors, or in separate compositions, and may be administered before, during, and/or after administration of the one or more STAT3 inhibitors.

In some embodiments of the methods and compositions of the invention, the one or more additional agents is an antiviral drug, a monoclonal antibody treatment, a steroid, COVID-19 convalescent plasma, or a combination of two or more of the foregoing. In some embodiments of the methods and compositions, the one or more additional agents includes a monoclonal antibody such as casirivimab, imdevimab, and/or bamlanivimab. In some embodiments, the one or more additional agents is an antiviral drug such as remdesivir, chloroquine, hydroxychloroquine, favipiravir, or a combination of two or more of the foregoing.

In some embodiments, the one or more additional agents is selected from the group consisting of amikacin, amphotericin formulations, atovaquone, any azole-containing anti-fungal drug, Bactrim, clindamycin, corticosteroids, echinocandin, fluconazole, flucytosine, itraconazole, posaconazole, quinine, sulfa drugs, trimethoprimsulfamethoxazole, voriconazole, baricitinib, interleukin-6 inhibitors, kinase inhibitors, tyrosine kinsase inhibitors, Tocilizumab, ivermectin, and any combination of two or more of the foregoing.

### Derivatives, Prodrugs, and Stereoisomers of STAT3 Inhibitors

Derivatives of parent molecules that retain STAT3 inhibitory activity (the same activity, or different STAT3 activity in type or extent) are known and can be utilized.

Derivatives of the STAT3 inhibitors exemplified herein can be synthesized by chemical transformations of the compounds' functional groups using standard chemical reactions. For example, these standard chemical reactions can include, but are not limited to: polar reactions under basic conditions, polar reactions under acidic conditions, pericyclic reactions, and free radical reactions. In another example, these standard chemical reactions can include, but are not limited to: addition reactions, substitution reactions, oxidation reactions, reduction reactions, elimination reactions, hydrolysis, acylation, amidations, etherification, and esterification. Alkane functional group transformations can include, but are not limited to: free radical chlorination (hv, Cl₂), free radical bromination (hv, Br₂), and allylic bromination (NBS). Alkene functional group transformations can include, but are not limited to: addition of HCl, addition of HBr, addition of HI, addition of H₃O(+), chlorination (Cl₂) bromination (Br₂), iodination (I₂), chlorohydrin formation (Cl₂/H₂O), bromohydrin formation (Br₂/H₂O), ether formation (H⁺/ROH), oxymercuration (Hg(OAc)₂/H₂O), oxymercuration, (Hg(OAc)₂/ROH), hydroboration, epoxidation (RCO₃H), dihydroxylation (OsO₄), dihydroxylation (KMnO₄), cyclopropanation, dichlorocyclopropanation, ozonolysis (reductive workup), ozonolysis (oxidative workup), oxidative cleavage (KMnO₄), hydrogenation, rearrangements (H shift), rearrangements (alkyl shift), free radical addition of HBr, and Sharpless epoxidation. Alkyne functional group transformations can include, but are not limited to: deprotonation (acetylide formation), S_{N}2 with alkyl halides, partial reduction (Lindlar), partial, reduction (Na/NH₃), hydroboration, oxymercuration, addition of HCl, HBr, or HI, addition of HCl, HBr, or HI, hydrogenation, ozonolysis, oxidative cleavage (KMnO₄), and halogenation (Cl₂, Br₂, I₂). The substitution reaction can include, but is not limited to: alcohol formation, nitrile formation, thiol formation, ether formation, thioether formation, azides, ester formation, acetylide addition, alkanes (Gilman reagents), ammonium salt formation, alkyl chloride formation, alkyl bromide formation, alkyl iodide formation, alkyl shift, and hydride shift. Elimination reactions can include, but are not limited to: alkenes from alkyl halides, alkenes from alcohols (strong acid), alkenes from alcohols (POCl₃), alkenes from alkyl halides, E1 with rearrangement (alkyl shift), Hoffmann elimination, and alkyne formation via elimination E1 with rearrangement (hydride shift). Organometallic reactions can include, but are not limited to: Grignard formation (alkyl halides), Grignard formation (alkenyl halides), reaction of Grignards with acids, addition of Grignards to aldehydes, addition of Grignards to ketones, addition of Grignards to esters, reaction of Grignards with CO₂, addition of Grignards to nitriles, formation of organolithium reagents, formation of Gilman reagents, S_{N}2 with Gilman reagents, addition of Gilman reagents to enones, addition of Gilman to acyl halides, Heck reaction, Suzuki reaction, and Stille reaction. Reactions of epoxides can include, but are not limited to: epoxide opening (basic conditions), epoxide opening (acidic conditions), epoxide opening (diol formation), epoxide formation (from halohydrins), epoxide formation (from alkenes), and Sharpless epoxidation of alkenes. Reactions of alcohols and thiols can include, but are not limited to: deprotonation (alkoxide formation), protonation (onium ion formation), conversion to tosylates/mesylates, conversion to alkyl chlorides (SOCl₂), conversion to alkyl bromides (PBr₃), oxidation to aldehydes (PCC), oxidation to ketones (PCC+others), oxidation to carboxylic acid, (H₂CrO₄+others), protection as silyl ethers, thiol formation (S_{N}2), and thiol oxidation to disulfides. Reactions of dienes can include, but are not limited to: Diels-alder reaction, polymerization of dienes, reactions of aromatics (arenes), nitration (HNO₃/H₂SO₄), chlorination (Cl₂ plus catalyst), bromination (Br₂ plus catalyst), sulfonylation (SO₃/H₂SO₄), Friedel Crafts alkylation (R-X plus catalyst), Friedel Crafts acylation (RCOX plus catalyst), iodination (I₂/catalyst), Side chain oxidation (KMnO₄), reduction of nitro groups, reduction of aromatic ketones, Side chain bromination, nucleophilic aromatic substitution (S_{N}Ar), and aryne formation (S_{N}Ar via arynes). Reactions of aldehydes and ketones can include, but are not limited to: hydrate formation (H₂O), cyanohydrin formation (CN), reduction of aldehydes (NaBH₄), reduction of aldehydes (LiAlH₄), reduction of ketones (NaBH₄), reduction of ketones (LiAlH₄), Grignard addition to aldehydes, Grignard addition to ketones, acetal formation (ROH/H⁺), acetal hydrolysis (H₃O⁺), imine, formation (RNH₂), Enamine formation (R₂NH), Wolff-Kishner: reduction to alkanes, Clemmensen, reduction to alkanes, oxidation to carboxylic acid (H₂CrO₄ or KMnO₄), keto-enol tautomerism, enolate formation, aldol addition reaction, alkylation of enolates, Wittig reaction (alkene formation), thioacetal formation, imine hydrolysis, oxidation to carboxylic acids (Tollens), haloform reaction, Baeyer-Villiger reaction, aldol condensation, Cannizarro reaction. Reactions of carboxylic acids can include, but are not limited to: deprotonation (carboxylate formation), formation via Grignard and CO₂, conversion to acid chloride (SOCl₂), reduction (LiAlH₄), Fischer esterification, and decarboxylation (of β-keto acids). Reactions of esters can include, but are not limited to: reduction to aldehydes (DIBAL-H), reduction to alcohols (LiAlH₄), hydrolysis to carboxylic acid (acidic), hydrolysis to carboxylic acid (basic), addition of Grignard reagents to esters, Claisen condensation, and transesterification (basic conditions). Reactions of acyl halides can include, but are not limited to: conversion to esters (ROH), conversion to carboxylic acids (H₂O), conversion to anhydrides (RCO₂), conversion to amides (RNH₂), conversion to ketones (Gilman reagents), and conversion to aldehydes (LiAlH(OtBu)₃). Reactions of α,β-unsaturated ketones (enones) can include, but are not limited to: Michael reaction (conjugate addition of enolates), conjugate addition of Gilman reagents, conjugate addition of other nucleophiles. Reactions of amines and amides can include, but are not limited to: dehydration of amides to nitriles (P₂O₅), Hofmann rearrangement, Gabriel synthesis of amines, reductive amination, formation of diazonium salts, reactions of diazonium salts, amide formation using DCC, amide formation from acid halides, and Curtius rearrangement. Reactions of nitriles can include, but are not limited to: addition of Grignard reagents to nitriles, reduction to amines (LiAlH₄), hydrolysis to carboxylic acids.

### RNA Virus Inhibitors

In certain embodiments, one or more RNA virus inhibitors can be administered with one or more STAT3 inhibitors and, optionally, with one or more IL-6 inhibitors and/or one or more JAK inhibitors. Additionally, one or more RNA virus inhibitors can be used in a composition with one or more STAT3 inhibitors and, optionally, with one or more IL-6 inhibitors and/or one or more JAK inhibitors.

RNA virus inhibitors can act as chain terminators, in which the RNA virus inhibitors can irreversibly stop RNA-dependent RNA polymerases from adding RNA bases to an RNA chain. Examples of chain terminating drugs that can be used in methods and compositions of the subject invention include remdesivir and derivatives of pyrazinacarboxamide, including favipiravir. Other RNA virus inhibitors that can be used in the subject invention include amantadine, rimantadine, DANA, FANA, zanamivir, oseltamivir, peramivir, viramidine, levoirin, mizoribine, EICAR, Ribavirin, VX-497, pleconaril, prodavir, VP-14637, JNJ-2408068, enfuvirtide, DAS181, BILN-2061,VX-950, ruprintrivir, 2′-c-methylcytidine, 2′C-methyl-7-deazaguanosine, 4′-azidocytidine, 2′-deoxy-2′-fluoroguanosine, flutamide, thiazolobenzimidazoles and derivatives thereof, RSV604, cyclosporine A, Debio-025, N-nonyl-deoxynojirimycin, mycophenolic acid, 3-deaza-adenosine, neplanocin A, 3-deazaneplanocin A, aristeromycin, pyrazofurin, 6-azauridine, and/or cyclopentylcytosine.

In certain embodiments, the RNA virus inhibitor can be used to treat comorbidities, complications, symptoms, or other consequences of a human coronavirus infection. Examples of complications include a cytokine storm, pneumonia, viral sepsis, acute respiratory distress syndrome, kidney failure, fibrosis of tissues, and cytokine release syndrome. Examples of symptoms include fever, cough, fatigue, shortness of breath, and loss of smell and/or taste.

### IL-6 Inhibitors

In certain embodiments, one or more IL-6 inhibitors can be administered with one or more STAT3 inhibitors and, optionally, with one or more RNA virus inhibitors and/or one or more JAK inhibitors. Additionally, one or more IL-6 inhibitors can be used in a composition with one or more STAT3 inhibitors and, optionally, with one or more RNA virus inhibitors and/or one or more JAK inhibitors.

IL-6 inhibitors can be directed against an IL-6 receptor or against IL-6. In preferred embodiments, IL-6 inhibitors are tocilizumab and/or sarilumab.

In certain embodiments, the Il-6 inhibitor can be used to treat comorbidities, complications, symptoms, or other consequences of a human coronavirus infection. Examples of complications include a cytokine storm, pneumonia, viral sepsis, acute respiratory distress syndrome, kidney failure, fibrosis of tissues, and cytokine release syndrome. Examples of symptoms include fever, cough, fatigue, shortness of breath, and loss of smell and/or taste.

### JAK Inhibitors

In certain embodiments, one or more JAK inhibitors can be administered or in a composition with one or more STAT3 inhibitors and, optionally, with one or more RNA virus inhibitors and/or one or more IL-6 inhibitors. Additionally, one or more JAK inhibitors can be used in a composition with one or more STAT3 inhibitors and, optionally, with one or more RNA virus inhibitors and/or one or more IL-6 inhibitors.

The JAK inhibitor can inhibit the JAK1 subtype, the JAK2 subtype, the JAK3 subtype, the TYK2 subtype or any combination of subtypes. In certain embodiments, the JAK inhibitor is Baricitinib, Ruxolitinib Oclacitinib Fedratinib, Upadacitinib, Filgotinib, Gandotinib, Lestaurtinib, Momelotinib, Pacritinib, Abrocitinib, Cerdulatinib, or any derivative thereof. In preferred embodiments, the JAK inhibitor is Baricitinib.

In certain embodiments, the JAK inhibitor can be used to treat comorbidities, complications, symptoms, or other consequences of a human coronavirus infection. Examples of complications include a cytokine storm, in which the innate immune system release a large number of cytokines, pneumonia, viral sepsis, acute respiratory distress syndrome, kidney failure, fibrosis of tissues, and cytokine release syndrome. Examples of symptoms include fever, cough, fatigue, shortness of breath, and loss of smell and/or taste.

### Compositions and Treatment

The STAT3 inhibitors of the present invention can be formulated into pharmaceutically acceptable salt forms or hydrate forms. Pharmaceutically acceptable salt forms include the acid addition salts and include hydrochloric, hydrobromic, nitric, phosphoric, carbonic, sulphuric, and organic acids like acetic, propionic, benzoic, succinic, fumaric, mandelic, oxalic, citric, tartaric, maleic, and the like. Pharmaceutically acceptable base addition salts include sodium, potassium, calcium, ammonium, and magnesium salts. Pharmaceutically acceptable salts of the polypeptides of the invention can be prepared using conventional techniques.

Administration of one or more STAT3 inhibitors and/or RNA virus inhibitors, IL-6 inhibitors, and/or JAK inhibitors can be carried out in the form of an oral tablet, capsule, or liquid formulation containing a therapeutically effective amount of the active ingredient (STAT3 inhibitor). Administration is not limited to oral delivery and includes intravascular (e.g., intravenous), intramuscular, or another means known in the pharmaceutical art for administration of active pharmaceutical ingredients.

Therapeutic or prophylactic application of the STAT3 inhibitors and/or RNA virus inhibitors, IL-6 inhibitors, and/or JAK inhibitors, and compositions containing them, can be accomplished by any suitable therapeutic or prophylactic method and technique presently or prospectively known to those skilled in the art. The STAT3 inhibitors and/or RNA virus inhibitors, IL-6 inhibitors, and/or JAK inhibitors can be administered by any suitable route known in the art including, for example, oral, sublingual, intramuscular, intraspinal, intracranial, nasal, rectal, parenteral, subcutaneous, or intravascular (e.g., intravenous) routes of administration. Administration of the STAT3 inhibitors of the invention can be continuous or at distinct intervals as can be readily determined by a person skilled in the art.

The STAT3 inhibitors and/or RNA virus inhibitors, IL-6 inhibitors, and/or JAK inhibitors, or pharmaceutical compositions of the present invention, can be administered as a single or as a divided dose. In some embodiments, the at least one initial dose can be single, divided, or a combination thereof In some embodiments, an amount of STAT3 inhibitors and/or RNA virus inhibitors, IL-6 inhibitors, and/or JAK inhibitors (e.g., 100 mg-1,000 mg) are to be administered 1, 2, 3, 4, or times per day, for 1, 2, 3, 4, 5, 6, 7, or more days. Treatment can continue as needed, e.g., for several weeks. Optionally, the treatment regimen can include a loading dose, with one or more daily maintenance doses. For example, in some embodiments, an initial loading dose in the range of 100 mg to 1,000 is administered, followed by a maintenance dose in the range of 100 mg to 1,000 mg every 12 hours for 1, 2, 3, 4, 5, 6, or 7, or more days. In some embodiments, an initial loading dose in the range of 200 mg to 600 mg is administered, followed by a maintenance dose in the range of 100 mg to 300 mg every 12 hours for a total of 9 doses.

STAT3 inhibitors and/or RNA virus inhibitors, IL-6 inhibitors, and/or JAK inhibitors and compositions comprising them can be formulated according to known methods for preparing pharmaceutically useful compositions. The STAT3 inhibitors and/or RNA virus inhibitors, IL-6 inhibitors, and/or JAK inhibitors can each be presented and administered in combination with one or more pharmaceutically acceptable carriers, excipients, and/or diluents as a pharmaceutical composition, optionally, with the addition of one or more additional agents.

Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive inhibitor is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject inhibitors include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of the inhibitors can also comprise suspension agents, protectants, lubricants, buffers, preservatives, and stabilizers. To provide for the administration of such dosages for the desired therapeutic treatment, in some embodiments, pharmaceutical compositions of the invention will advantageously comprise between about 0.1% and 45%, and especially, 1 and 15% by weight of the total of one or more of the inhibitor based on the weight of the total composition including carrier or diluent.

The STAT3 inhibitors and/or RNA virus inhibitors, IL-6 inhibitors, and/or JAK inhibitors of the subject invention can also be administered utilizing liposome technology, slow release capsules, implantable pumps, and biodegradable containers. These delivery methods can, advantageously, provide a uniform dosage over an extended period of time.

The STAT3 inhibitors and/or RNA virus inhibitors, IL-6 inhibitors, and/or JAK inhibitors can also be modified by the addition of chemical groups, such as PEG (polyethylene glycol). PEGylated polypeptides typically generate less of an immunogenic response and exhibit extended half-lives in vivo in comparison to polypeptides that are not PEGylated when administered in vivo. Methods for PEGylating proteins and polypeptides known in the art (see, for example, U.S. Pat. No. 4,179,337). The subject polypeptides and polynucleotides can also be modified to improve cell membrane permeability. In one embodiment, cell membrane permeability can be improved by attaching a lipophilic moiety, such as a steroid, to the inhibitor. Other groups known in the art can be linked to the STAT3 inhibitors.

The subject invention also concerns a packaged dosage formulation comprising in one or more packages, packets, or containers at least one STAT3 inhibitor and/or RNA virus inhibitor, IL-6 inhibitor, and/or JAK inhibitor, and/or composition of the subject invention formulated in a pharmaceutically acceptable dosage. The package can contain discrete quantities of the dosage formulation, such as tablet, capsules, lozenge, and powders. The quantity of STAT3 inhibitor in a dosage formulation and that can be administered to a patient can vary from about 1 mg to about 5000 mg, or about 1 mg to about 2000 mg, or more typically about 1 mg to about 500 mg, or about 5 mg to about 250 mg, or about 10 mg to about 100 mg. In some embodiments, the amount is in the range of 100 mg to 600 mg, to be administered 1, 2, 3, or 4 times per day, for 2, 3, 4, 5, 6, 7 or more days.

The subject invention also concerns kits comprising in one or more containers an STAT3 inhibitor and/or RNA virus inhibitors, IL-6 inhibitors, and/or JAK inhibitors of the present invention. A kit of the invention can also comprise one or more compounds, biological molecules, or drugs. In one embodiment, a kit of the invention comprises an STAT3 inhibitor, and optionally comprises one or more of a drug or composition used in treating a viral infection, such RNA virus inhibitors, IL-6 inhibitors, and/or JAK inhibitors.

Optionally, the methods further comprise, prior to administering the STAT3 inhibitor and/or RNA virus inhibitors, IL-6 inhibitors, and/or JAK inhibitors to the subject, identifying the subject as having a human coronavirus infection (human coronavirus, generally, or a specific strain of coronavirus, such as SARS-CoV-2), or not having a human coronavirus infection. If the subject is identified as having a human coronavirus infection, the STAT3 inhibitor and/or RNA virus inhibitors, IL-6 inhibitors, and/or JAK inhibitors can be administered to the human subject as therapy. If the human subject is identified as not having a human coronavirus infection, the STAT3 inhibitor and/or RNA virus inhibitors, IL-6 inhibitors, and/or JAK inhibitors can be withheld, or the STAT3 inhibitor can be administered as prophylaxis, or an alternative agent can be given. The identifying step may comprise assaying a biological sample (e.g., blood, saliva, or urine) obtained from the subject for the presence of human coronavirus nucleic acids or human coronavirus proteins, such as SARS-CoV-2 nucleic acids or proteins. In some embodiments, assaying includes the use of reverse transcriptase-polymerase chain reaction (RT-PCR), immunological assay (e.g., ELISA), or Plaque-reduction neutralization testing (PRNT).

Thus, optionally, the methods include, prior to administration of the STAT3 inhibitor and/or RNA virus inhibitors, IL-6 inhibitors, and/or JAK inhibitors, or re-administration of the STAT3 inhibitor and/or RNA virus inhibitors, IL-6 inhibitors, and/or JAK inhibitors, determining whether the subject has a human coronavirus infection or one or more symptoms consistent with a human coronavirus infection. Some individuals infected with coronavirus will not know they have the infection because they will not have symptoms.

In some embodiments of the methods of the invention, the human coronavirus is selected from among SARS-CoV-2 or a variant thereof, SARS-CoV, and MERS-CoV. SARS-CoV-2 is a novel human coronavirus that causes coronavirus disease 2019, also known as COVID-19 and COVID19. MERS-CoV is the beta coronavirus that causes Middle East Respiratory Syndrome, or MERS. SARS-CoV is the beta coronavirus that causes severe acute respiratory syndrome, or SARS.

In some embodiments of the methods of the invention, the human coronavirus is a common human coronavirus, such as type 229E (an alpha coronavirus), NL63 (an alpha coronavirus), OC43 (a beta coronavirus), and HKU1 (a beta coronavirus).

The symptoms of a coronavirus infection depend on the type of coronavirus and severity of the infection. If a subject has a mild to moderate upper-respiratory infection, such as the common cold, symptoms may include: runny nose, headache, cough, sore throat, fever, and general feeling of being unwell. Some coronaviruses can cause severe symptoms. These infections may turn into bronchitis and pneumonia, which can cause symptoms such as fever (which can be quite high with pneumonia), cough with mucus, shortness of breath, and chest pain or tightness when the subject breaths or coughs.

The clinical spectrum of SARS-CoV-2 may range from mild disease with non-specific signs and symptoms of acute respiratory illness, to severe pneumonia with respiratory failure and septic shock. Asymptomatic infections have also been reported.

To diagnose coronavirus infections, healthcare providers typically take the subject's medical history and ask the subject their symptoms, do a physical examination, and may conduct laboratory tests on a biological sample such as blood, or a respiratory specimen such as sputum or a throat swab.

In some embodiments, a molecular assay may be used to detect the presence or absence of human coronavirus in a biological sample from the subject. For example, several assays that detect SARS-CoV-2 have been under development. Some assays may detect only the novel virus and some may also detect other strains (e.g., SARS-CoV) that are genetically similar. The table below is a summary of some available protocols and their gene targets.

| Country | Institution | Gene targets |
|---|---|---|
| United States | US CDC | Three N primers, |
| | | RdRP |
| China | China CDC | ORF1ab and N |
| Germany | Charité | RdRP, E, N |
| Hong Kong | HKU | ORF1b-nsp14, N |
| Japan | National Institute of | Pancorona and |
| | Infection Diseases, | multiple targets, |
| | Department of | Spike protein |
| | Virology III | |
| Thailand | National Institute of | N |
| | Health | |

China CDC Primers and probes for detection 2019-nCoV (24 Jan. 2020)
Diagnostic detection of Wuhan coronavirus 2019 by real-time RT-PCR—Charité, Berlin Germany (17 Jan. 2020)
Detection of 2019 novel coronavirus (2019-nCoV) in suspected human cases by RT-PCR—Hong Kong University (23 Jan. 2020)
PCR and sequencing protocol for 2019-nCoV—Department of Medical Sciences, Ministry of Public Health, Thailand (Updated 28 Jan. 2020)
PCR and sequencing protocols for 2019-nCo—National Institute of Infectious Diseases Japan (24 Jan. 2020)
US CDC Real-Time RT-PCR Panel for Detection 2019-Novel Coronavirus (28 Jan. 2020)
US CDC panel primer and probes—U.S. CDC, USA (28 Jan. 2020)
(“WHO interim guidance for laboratory testing for 2019 novel coronavirus (2019-nCoV) in humans” from World Health Organization website).

SARS-CoV-2 RNA has been detected from upper and lower respiratory tract specimens, and the virus has been isolated from upper respiratory tract specimens and bronchoalveolar lavage fluid. SARS-CoV-2 RNA has been detected in blood and stool specimens. The duration of SARS-CoV-2 RNA detection in the upper and lower respiratory tracts and in extrapulmonary specimens has not been determined. It is possible that RNA could be detected for weeks, which has occurred in some cases of MERS-CoV or SARS-CoV infection. Viable SARS-CoV has been isolated from respiratory, blood, urine, and stool specimens, and viable MERS-CoV has been isolated from respiratory tract specimens.

Treatment methods optionally include steps of advising that the subject get plenty of rest and drink fluids for hydration and administration of agents that alleviate symptoms of coronavirus infection, such as those that reduce fever and pain (e.g., acetaminophen and/or paracetamol), particularly for common human coronavirus infections. The methods may include administration of the fluids to the subject for hydration.

The subject may be any age or gender. In some cases, the subject may be an infant or older adult. In some embodiments, the subject is 40 years of age or older. In some embodiments, the subject is 55 years of age or older. In some embodiments, the subject is 60 years of age or older. In some embodiments, the subject is a child. In some embodiments, the subject is an infant. In some embodiments, the subject (of any age or gender) has heart or lung disease, diabetes, or a weakened immune system.

In some embodiments, the subject has cancer at the time of administration of the STAT3 inhibitor and/or RNA virus inhibitors, IL-6 inhibitors, and/or JAK inhibitors. In other embodiments, the subject does not have cancer at the time of administration of the STAT3 inhibitor and/or RNA virus inhibitors, IL-6 inhibitors, and/or JAK inhibitors.

The invention further provides kits, including STAT3 inhibitors and/or RNA virus inhibitors, IL-6 inhibitors, and/or JAK inhibitors and pharmaceutical formulations, packaged into suitable packaging material, optionally in combination with instructions for using the kit components, e.g., instructions for performing a method of the invention. In one embodiment, a kit includes an amount of an STAT3 inhibitor and/or RNA virus inhibitors, IL-6 inhibitors, and/or JAK inhibitors, and instructions for administering the inhibitor to a subject in need of treatment on a label or packaging insert. In further embodiments, a kit includes an article of manufacture, for delivering the inhibitor into a subject locally, regionally, or systemically, for example.

As used herein, the term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating a human coronavirus infection, an assay for identifying a subject having a human coronavirus infection, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, pharmaceutical compositions can be included in a container, pack, or dispenser together with instructions for administration to a subject to treat a human coronavirus infection. Instructions may additionally include appropriate administration route, dosage information, indications of a satisfactory clinical endpoint or any adverse symptoms that may occur, storage information, expiration date, or any information required by regulatory agencies such as the Food and Drug Administration or European Medicines Agency for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Kits can additionally include a buffering agent, a preservative, or an agent for stabilizing the STAT3 inhibitor and/or RNA virus inhibitors, IL-6 inhibitors, and/or JAK inhibitors. The kit can also include control components for assaying for the presence of human coronavirus, e.g., a control sample or a standard. Each component of the kit can be enclosed within an individual container or in a mixture and all of the various containers can be within single or multiple packages.

### Definitions

As used herein, a subject (preferably, a human) is “in need of” a treatment if such subject would benefit biologically, medically or in quality of life from such treatment. In some embodiments, the subject has a coronavirus infection and is in need of therapy. In other embodiments, the subject does not have a coronavirus infection and is in need of prophylaxis. In some embodiments, the subject in need of prophylaxis is at risk of becoming infected with the coronavirus. In some embodiments, the subject is at increased risk of becoming infected with the coronavirus relative to others in the population. In some embodiments, the subject has a coronavirus infection and is symptomatic. In some embodiments, the subject has a coronavirus infection and is asymptomatic.

As used herein, the terms “subject”, “patient”, and “individual” refer to a human or non-human animal of any age or gender. In some embodiments, the subject is a mammal, preferably a human.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to prophylaxis (preventing or delaying the onset or development or progression of the disease or disorder).

As used herein, the term “administration” is intended to include, but is not limited to, the following delivery methods: topical, oral, sublingual, parenteral, subcutaneous, transdermal, transbuccal, intravascular (e.g., intravenous or intra-arterial), intramuscular, subcutaneous, intranasal, and intra-ocular administration. Administration can be local at a particular anatomical site, such as a site of infection, or systemic.

As used herein, “asymptomatic” refers to a human subject that may or may not have been exposed to a coronavirus, such as a variant of SARS-CoV-2, and does not present symptoms related to a coronavirus infection, such as a SARS-CoV-2 infection. An asymptomatic human subject may or may not be infected with a coronavirus, such as SARS-CoV-2. For example, an asymptomatic human subject includes a human subject that has no symptoms related to a SARS-CoV-2 infection and has been in close contact with someone that is infected with SARS-CoV-2. In another example, an asymptomatic human subject includes a human subject that has no symptoms related to a SARS-CoV-2 infection and has tested positive for infection of SARS-CoV-2.

As used herein, the meaning of the phrase “close contact” in the context of a human having been exposed to someone infected with a coronavirus (e.g., SARS-CoV-2 or variant) will depend upon the exposure setting. The meaning may be the definition adopted by the government health agency having jurisdictional authority, and may be based on factors such as the presence of special populations. In some embodiments, close contact exists if the subj ect was within 6 feet of an infected person for a cumulative total of 15 minutes or more over a 24-hour period.

As used herein, the term “contacting” in the context of contacting a cell with at least one STAT3 inhibitor in vitro or in vivo means bringing at least one inhibitor into contact with the cell, or vice-versa, or any other manner of causing the inhibitor and the cell to come into contact.

The compounds of the present invention can be formulated into pharmaceutically-acceptable salt forms. Pharmaceutically-acceptable salts of the compounds of the invention can be prepared using conventional techniques. “Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, a “derivative” or “pharmaceutically active derivative” refers to any compound that upon administration to the recipient, is capable of providing directly or indirectly, the activity disclosed herein (e.g., anti-coronavirus activity and/or STAT3 inhibitory activity, such as inhibition of STAT3). The term “indirectly” also encompasses “prodrugs” which may be converted to the active form of the drug, e.g., via endogenous enzymes or metabolism (biotransformation). The prodrug is a derivative of the compounds according to the invention and presenting STAT3 inhibitory activity that has a chemically or metabolically decomposable group, and a compound that may be converted into a pharmaceutically active compound according to the invention in vivo by solvolysis under physiological conditions. The prodrug is converted into a compound according to the present invention by a reaction with an enzyme, gastric acid or the like under a physiological condition in the living body, e.g., by oxidation, reduction, hydrolysis or the like, each of which is carried out enzymatically. These compounds can be produced from compounds of the present invention according to well-known methods. The term “indirectly” also encompasses metabolites of compounds according to the invention. Chemical reactions, reactants, and reagents useful for making derivatives can be found, for example, in March's Advanced Organic Chemistry, 7^{th} edition, 2013, Michael B. Smith, which is incorporated herein by reference in its entirety.

More specifically, the term “prodrug” refers to a chemical compound that can be converted by the body (i.e., biotransformed) to another chemical compound that has pharmacological activity. The prodrug may itself have pharmacological activity before conversion, or be inactive before conversion and activated upon conversion. Active prodrugs or inactive prodrugs of compounds of the invention may be administered to a subject or contacted with a cell in vitro or in vivo. Instead of administering a drug directly, a prodrug may be used instead to improve how a drug is absorbed, distributed, metabolized, and excreted (ADME). For example, a prodrug may be used to improve bioavailability when a drug itself is poorly absorbed from the gastrointestinal tract, or to improve how selectively the drug interacts with cells or processes that are not its intended target, which can reduce adverse or unintended effects of a drug. Major types of prodrugs include, but are not limited to, type I prodrugs, which are biotransformed inside cells (intracellularly), and type II prodrugs, which are biotransformed outside cells (extracellularly), such as in digestive fluids or in the body's circulatory system. These types can be further categorized into subtypes based on factors such as whether the intracellular bioactivation location is also a site of therapeutic action, or whether or not bioactivation occurs in the gastrointestinal fluids or in the circulation system (Wu, Kuei-Meng, “A New Classification of Prodrugs: Regulatory Perspectives, Pharmaceuticals, 2009, 2(3):77-81, which is incorporated by reference herein in its entirety).

The term “metabolite” refers to all molecules derived from any of the compounds according to the present invention in a cell or organism, preferably mammal. Pharmaceutically active metabolites of the compounds of the invention may be administered to a subject or contacted with a cell in vitro or in vivo.

The phrase “pharmaceutically acceptable” indicates that the substance or composition must be compatible chemically and/or toxicologically, with the other ingredients comprising a formulation, and/or the mammal being treated therewith.

Pharmaceutical formulations include “pharmaceutically acceptable” and “physiologically acceptable” carriers, diluents or excipients. In this context, the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the compositions.

The phrase “effective amount” means an amount of an agent, such as a STAT3 inhibitor, that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, the term “inhibit”, “inhibition” or “inhibiting” refers to the reduction or suppression of a given condition, symptom, or disorder, or disease (e.g., coronavirus infection, or coronavirus viral load or titer), or a significant decrease in the baseline activity of a biological activity or process (e.g., STAT3 production and inhibitors of STAT3 activity).

As used herein, the terms “inhibitor of STAT3” and “STAT3 inhibitor” are interchangeable and refer to any agent capable of reducing or disrupting STAT3 signaling. Exemplary classes of STAT3 inhibitors include, but are not limited to, small molecules, and macromolecules or biologics such as anti-STAT3 antibodies (monoclonal or polyclonal antibodies, or antigen-binding fragments thereof), inhibitory polynucleotides or oligonucleotides that reduce STAT3 transcription or translation (e.g., antisense, siRNA, shRNA targeting STAT3). Therefore, “inhibition” or “inhibiting” of STAT3 encompasses both pharmacological blocking of STAT3 and genetic deletion of or interference with STAT3 coding sequences. A “STAT3 inhibitor” includes any agent capable of disrupting STAT3 signaling in a cell. Suitable STAT3 inhibitors include not only agents that directly interfere with binding of STAT3 to its consensus sequence, but also may include agents that interfere with phosphorylation (e.g., tyrosine or serine phosphorylation) required for STAT3 dimerization by, e.g., inhibiting tyrosine kinases and/or SH2-pY interactions. Further suitable agents may interfere with nuclear transport of STAT3 dimers. In addition, structural features of STAT3, such as the DNA binding and transactivation domains, may suitably serve as targets for functional disruption of STAT3. The most suitable agents for inhibiting STAT3 will be those that are potent and selective disruptors of STAT3 signaling activity. The STAT3 inhibitor(s) may be in an isolated or un-isolated form and optionally placed in a pharmaceutical composition comprising the STAT3 inhibitor(s) and a pharmaceutically acceptable carrier, excipient, and/or diluent, for administration to a subject. The STAT3 inhibitor(s) may be naturally occurring or artificially created.

As used herein, the terms “inhibitor of JAK” and “JAK inhibitor” are interchangeable and refer to any agent capable of reducing or disrupting JAK pathway signaling. Exemplary classes of JAK inhibitors include, but are not limited to, small molecules, and macromolecules or biologics such as anti-JAK pathway member antibodies (monoclonal or polyclonal antibodies, or antigen-binding fragments thereof), inhibitory polynucleotides or oligonucleotides that reduce JAK pathway member transcription or translation (e.g., antisense, siRNA, shRNA targeting JAK pathway member). Therefore, “inhibition” or “inhibiting” of JAK encompasses both pharmacological blocking of members of the JAK signaling pathway and genetic deletion of or interference with coding sequences of members of the JAK signaling pathway. The JAK inhibitor(s) may be in an isolated or un-isolated form and optionally placed in a pharmaceutical composition comprising the JAK inhibitor(s) and a pharmaceutically acceptable carrier, excipient, and/or diluent, for administration to a subject. The JAK inhibitor(s) may be naturally occurring or artificially created.

As used herein, the term “RNA virus inhibitor” refers to agents that inhibit RNA virus replication, host cell entry, and/or enhance host defense systems. For example, the RNA virus inhibitor may intervene in the steps of viral infection, including viral attachment, fusion/endocytosis, replication, assembly and budding, and/or target the viral envelope. The RNA virus inhibitor may target the cellular machineries of defense, programmed cell death, and/or metabolism. Exemplary classes of RNA virus inhibitors include, but are not limited to, small molecules, and macromolecules or biologics such as antibodies (monoclonal or polyclonal antibodies, or antigen-binding fragments thereof), inhibitory polynucleotides or oligonucleotides that reduce transcription or translation (e.g., antisense, siRNA, shRNA). The RNA virus inhibitor(s) may be in an isolated or un-isolated form and optionally placed in a pharmaceutical composition comprising the RNA virus inhibitor(s) and a pharmaceutically acceptable carrier, excipient, and/or diluent, for administration to a subject. The RNA virus inhibitor(s) may be naturally occurring or artificially created.

As used herein, the terms “IL-6 inhibitor” and “Anti-IL-6 agent” are used interchangeably to refer to agents that inhibit IL-6 from binding to a receptor or inhibit an IL-6 receptor, or which cause a decrease in IL-6 production or in baseline IL-6 activity. Exemplary classes of IL-6 inhibitors include, but are not limited to, small molecules, and macromolecules or biologics such as antibodies (monoclonal or polyclonal antibodies, or antigen-binding fragments thereof), inhibitory polynucleotides or oligonucleotides that reduce transcription or translation (e.g., antisense, siRNA, shRNA). Exemplary IL-inhibitors include, but are not limited to, sarilumab and tocilizumab. The IL-6 inhibitor(s) may be in an isolated or un-isolated form and optionally placed in a pharmaceutical composition comprising the IL-6 inhibitor(s) and a pharmaceutically acceptable carrier, excipient, and/or diluent, for administration to a subject. The IL-6 inhibitor(s) may be naturally occurring or artificially created.

As used herein, the terms “preventing” or “prevention” refers to achieving, partially, substantially, or completely, one or more of the following results: avoiding the disease, disorder, or syndrome resulting from a coronavirus infection, such as an infection from SARS-CoV-2 or a variant, such disease as COVID-19; avoiding clinical symptom or indicator associated with a disease, disorder, or syndrome resulting from a coronavirus infection, such as infection from SARS-CoV-2 or a variant; reducing the severity of the disease, disorder, or syndrome resulting from a coronavirus infection, such as from an infection of SARS-CoV-2 or a variant; or avoiding a coronavirus infection, such as an infection from SARS-CoV-2 or a variant.

The terms “strain” and “variant” in the context of viruses are used interchangeably herein to refer to subtypes of a virus that are genetically distinct from each other. For example, SARS-CoV-2 has multiple variants currently circulating globally. Such SARS-CoV-2 variants include at least B.1.1.7 identified in the United Kingdom, B.1.351 identified in South Africa, and P.1 identified in travelers from Brazil. For example, SARS-CoV-2 variants may include mutations, such as the following: E484K, which was first discovered in the United Kingdom; L452R, which was detected in Denmark; and D614G discovered in China in January 2020. Other mutations identified in SARS-CoV-2 variants include, for example, the 69/70 deletion, 144Y deletion, N501Y, A570D, P681H, E484K, and K417N/T.

The terms “compounds of the present invention” or “agents of the invention” (unless specifically identified otherwise) refer to STAT3 inhibitors, JAK inhibitors, RNA virus inhibitors, and/or IL-6 inhibitors including salts thereof, as well as all stereoisomers (including diastereoisomers and enantiomers), rotamers, tautomers and isotopically labeled compounds (including deuterium substitutions), as well as inherently formed moieties (e.g., polymorphs, solvates and/or hydrates). For purposes of this invention, solvates and hydrates are generally considered compositions.

The term “a,” “an,” “the” and similar terms used in the context of the present invention (especially in the context of the claims) are to be construed to cover both the singular and plural unless otherwise indicated herein or clearly contradicted by the context. For example, the term “cell” includes a singular cell and a plurality of cells unless specified to the contrary; and the term “inhibitor” includes a singular inhibitor and a plurality of inhibitors.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A method for treating or preventing human coronavirus infection, or a symptom thereof, in a human subject, said method comprising administering an effective amount of a STAT3 inhibitor to the human subject.

2. The method of claim 1, wherein the coronavirus is SARS-CoV-2 or a variant thereof, SARS-CoV or a variant thereof, or MERS-CoV or a variant thereof.

3-4. (canceled)

5. The method of claim 1, wherein the human coronavirus is a common human coronavirus selected from the group consisting of 229E, NL63, OC43, and HKU1.

6. The method of claim 1, wherein the symptom is fibrosis of a tissue, a cytokine storm, and/or a symptom of COVID-19.

7. The method of claim 6, wherein the tissue is a lung tissue.

8. (canceled)

9. The method of claim 1, wherein the human subject has the coronavirus infection at the time of said administering or has previously had the coronavirus infection at the time of said administering.

10. (canceled)

11. The method of claim 9, further comprising, prior to said administering, identifying the subject as having the coronavirus infection, wherein said identifying comprises assaying a biological sample obtained from the subject for the presence of coronavirus nucleic acid or coronavirus protein.

12. The method of claim 1, further comprising determining that the human subject is infected with a coronavirus prior to or contemporaneously with initiating said administering.

13-15. (canceled)

16. The method of claim 1, wherein the human subject does not have the coronavirus infection at the time of said administering, and the STAT3 inhibitor is administered as prophylaxis, wherein the STAT3 inhibitor is administered orally, sublingually, intravenously, intravascularly, nasally, rectally, parenterally, subcutaneously, or intramuscularly.

17-18. (canceled).

19. The method of claim 1, wherein the STAT3 inhibitor is one or more of Pt-401 (GLG-401), Withacnistin (GLG-101), NSC-74859 (GLG-302), NSC-59263 (GLG-303), NSC-42067 (GLG-304), GLG-305, S31-M2001 (GLG-202), HL2-006-1 (GLG-306), HL2-006-2 (GLG-307), Pyrimethamine (GLG-801), Pimozide (GLG-802), Guanabenz Acetate (GLG-803), Alprenolol hydrochloride (GLG-804), Solanine alpha (GLG-806), Fluoxetine hydrochloride (GLG-807), Ifosfamide (GLG-808), Pyrvinium pamoate (GLG-809), Moricizine hydrochloride (GLG-810), 3,3′-oxybis[tetrahydrothiophene, 1,1,1′,1′-tetraoxide] (GLG-811), Nifuroxazide (GLG-812), Pyrimethamine IV formulae—methane sulfonic acid salt (GLG-805), 188-9 (TTi-101) N-{1′,2-dihydroxy-[1,2′-binaphthalen]-4′-yl}-4-methoxybenzene-1-sulfonamide, STX-0119, Napabucasin (BBI-608), Niclosamide, CPD-188, GPB730, GPA 512, WP1066, Curcumin, SBT-100, SBT-102, SBT-300, or a pharmaceutically acceptable salt, derivative, or prodrug thereof.

20. The method of claim 1, further comprising administering an additional agent for treating or preventing coronavirus infection, or a symptom thereof, in the same formulation as the STAT3 inhibitor, or in a separate formulation before, during, or after administration of the STAT3 inhibitor.

21. The method of claim 20, wherein the additional agent is one or more RNA virus inhibitors, one or more IL-6 inhibitors, one or more Janus kinase (JAK) inhibitors, or a combination of two or more of the foregoing.

22. The method of claim 21, wherein the JAK inhibitor is Baricitinib; or wherein the RNA virus inhibitor is remdesivir, favipiravir, or a combination thereof or wherein the IL-6 inhibitor is tocilizumab, sarilumab, or a combination thereof.

23-24. (canceled)

25. The method of claim 20, wherein the additional agent is an antiviral drug, a monoclonal antibody treatment, a steroid, COVID-19 convalescent plasma, or a combination of two or more of the foregoing.

26. The method of claim 25, wherein the monoclonal antibody treatment is casirivimab, imdevimab, bamlanivimab, or a combination of two or more of the foregoing; or wherein the antiviral drug is remdesivir, chloroquine, hydroxychloroquine, favipiravir, or a combination of two or more of the foregoing.

27. (canceled)

28. The method of claim 20, wherein the additional agent is selected from the group consisting of amikacin, amphotericin formulations, atovaquone, any azole-containing anti-fungal drug, Bactrim, clindamycin, corticosteroids, echinocandin, fluconazole, flucytosine, itraconazole, posaconazole, quinine, sulfa drugs, trimethoprimsulfamethoxazole, voriconazole, baricitinib, interleukin-6 inhibitors, kinase inhibitors, tyrosine kinsase inhibitors, Tocilizumab, ivermectin, and any combination of two or more of the foregoing.

29. The method of claim 1, wherein the STAT3 inhibitor inhibits phosphorylation of STAT3 at serine 727 (e.g., pyrimethamine (GLG-801) or a pharmaceutically acceptable salt thereof, such as pyrimethamine methanesulfonate (GLG-805)); or wherein the STAT3 inhibitor inhibits phosphorylation of STAT3 at tyrosine 705 (e.g., NSC-74859 (GLG-302) or GLG-305, or a pharmaceutically acceptable salt thereof); or wherein a combination of STAT3 inhibitors is administered to the subject, wherein at least one STAT3 inhibitor of the combination inhibits phosphorylation of STAT3 at serine 727, and wherein at least one STAT3 inhibitor of the combination inhibits phosphorylation of STAT3 at tyrosine 705.

30-31. (canceled).

32. The method of claim 1, wherein the STAT3 inhibitor increases NO production; or wherein the STAT3 inhibitor reduces STAT3-induced prevention of cell division; or wherein the STAT3 inhibitor reduces the STAT3-induced activation of TC45 phosphatase.

33-34. (canceled)

35. A composition of matter, comprising:
(a) packaged dosage formulation comprising a STAT3 inhibitor, in a pharmaceutically acceptable dosage in one or more packages, packets, or containers; and instructions for administering the STAT3 inhibitor, to treat or prevent human coronavirus infection; or
(b) a kit comprising, in one or more containers, STAT3 inhibitor; and instructions for administering the STAT3 inhibitor to treat or prevent human coronavirus infection.

36. The composition of matter of claim 35, wherein the STAT3 inhibitor of the packaged dosage formulation is a combination of STAT3 inhibitors, wherein at least one STAT3 inhibitor of the combination inhibits phosphorylation of STAT3 at serine 727, and wherein at least one STAT3 inhibitor of the combination inhibits phosphorylation of STAT3 at tyrosine 705; or wherein the STAT3 inhibitor of the kit is a combination of STAT3 inhibitors, wherein at least one STAT3 inhibitor of the combination inhibits phosphorylation of STAT3 at serine 727, and wherein at least one STAT3 inhibitor of the combination inhibits phosphorylation of STAT3 at tyrosine 705.

37. (canceled)

38. The composition of matter of claim 35, wherein the kit further comprises a component for testing for the presence of a human coronavirus infection in a biological sample.

39. The composition of matter of claim 35, wherein the packaged dosage formulation further comprises one or more RNA virus inhibitors, one or more IL-6 inhibitors, one or more Janus kinases (JAK) inhibitors, or a combination of two or more of the foregoing; and instructions for administering the one or more RNA virus inhibitors, instructions for administering the one or more IL-6 inhibitors and/or instructions for administering the one or more JAK inhibitors; or wherein the kit further comprises one or more RNA virus inhibitors, one or more IL-6 inhibitors, one or more Janus kinases (JAK) inhibitors, or a combination of two or more of the foregoing; and instructions for administering the one or more RNA virus inhibitors, instructions for administering the one or more IL-6 inhibitors and/or instructions for administering the one or more JAK inhibitors.

40. (canceled)
